(19) United States
(12) Patent Application Publication
(10) Pub. No.: US 2020/0261734 A1
(43) Pub. Date: Aug. 20, 2020

(54) LEAD-IN-LEAD SYSTEMS AND METHODS FOR CARDIAC THERAPY

(71) Applicant: Medtronic, Inc., Minneapolis, MN (US)

(72) Inventors: Zhongping Yang, Woodbury, MN (US); Eduardo N. Warman, Maple Grove, MN (US); Manfred Justen, Lino Lakes, MN (US)

(21) Appl. No.: 16/780,083

(22) Filed: Feb. 3, 2020

Related U.S. Application Data
(60) Provisional application No. 62/805,770, filed on Feb. 14, 2019.

(51) Int. Cl. A61N 1/375; A61N 1/362; A61N 1/368; A61N 1/05; A61N 1/372

(52) U.S. Cl. CPC A61N 1/37516; A61N 1/362; A61B 17/3468; A61N 1/0573; A61N 1/37205; A61N 1/3684

(57) ABSTRACT

A lead-in-lead system may include a first implantable lead having a first electrode and a second implantable lead having a second electrode guided by the first implantable lead to an implantation site. The second electrode may be implanted in a patient's heart distal to the first electrode at the same implantation site or at a second implantation site. Various methods may be used to deliver the lead-in-lead system to one or more implantation sites including at the triangle of Koch for ventricle-from-atrium (VfA) therapy, at the right ventricular septal wall for dual bundle-branch pacing, or in the coronary vasculature for left side sensing and pacing.

### FIELD

The present technology is generally related to implantable medical devices, systems, and methods. In particular, the present technology is related to implantable lead delivery for cardiac sensing and therapy.

### BACKGROUND

Implantable medical devices (IMDs), such as cardiac pacemakers or implantable cardioverter defibrillators, deliver therapeutic stimulation to patients' hearts. Patients with a conduction system abnormality, such as poor atrioventricular (AV) node conduction or poor sinoatrial (SA) node function, may receive an IMD, such as a pacemaker, to restore a more normal heart rhythm and AV synchrony. Some types of IMDs, such as cardiac pacemakers, implantable cardioverter-defibrillators (ICDs), or cardiac resynchronization therapy (CRT) devices, provide therapeutic electrical stimulation to a heart of a patient via electrodes on one or more implantable endocardial, epicardial, or coronary venous leads that are positioned in or adjacent to the heart. The therapeutic electrical stimulation may be delivered to the heart in the form of pulses or shocks for pacing, cardioversion, or defibrillation. In some cases, an IMD may sense intrinsic depolarizations of the heart and control the delivery of therapeutic stimulation to the heart based on the sensing.

Existing pacing techniques involve pacing one or more of the four chambers of patient's heart 12—left atrium (LA) 33, right atrium (RA) 26, left ventricle (LV) 32 and right ventricle (RV) 28, all of which are shown in the anterior view of a frontal section of patient's heart 12 illustrated in FIG. 1. Some therapeutic pacing techniques involve the cardiac conduction system. The cardiac conduction system, like a “super highway,” may be described as quickly conducting electrical pulses whereas pacing cardiac muscle tissue may slowly conduct electrical pulses, like “traveling on a dirt road.” The cardiac conduction system includes SA node 1, atrial internodal tracts 2, 4, 5 (i.e., anterior internodal 2, middle internodal 4, and posterior internodal 5), atrioventricular node (AV node) 3, His bundle 13 (also known as the atrioventricular bundle or bundle of His), and bundle branches including the left bundle branch (LBB) 8a and the right bundle branch (RBB) 8b. FIG. 1 also shows the arch of aorta 6 and Bachman's bundle 7. The SA node, located at the junction of the superior vena cava and right atrium, is considered to be the natural pacemaker of the heart since it continuously and repeatedly emits electrical impulses. The electrical impulse spreads through the muscles of RA 26 to LA 33 to cause synchronous contraction of the atria. Electrical impulses are also carried through atrial internodal tracts to AV node 3—the sole connection between the atria and the ventricles.

Conduction through the AV nodal tissue takes longer than through the atrial tissue, resulting in a delay between atrial contraction and the start of ventricular contraction. The AV delay, which is the delay between atrial contraction and ventricular contractor, allows the atria to empty blood into the ventricles. Then, the valves between the atria and ventricles close before causing ventricular contraction via branches of the bundle of His.

His bundle 13 is located in the membranous atrioventricular septum near the annulus of the tricuspid valve. His bundle 13 splits into right and left bundle branches 8a, 8b and are formed of specialized fibers called “Purkinje fibers” 9. Purkinje fibers 9 may be described as rapidly conducting an action potential down the ventricular septum, spreading the depolarization wavefront quickly through the remaining ventricular myocardium, and producing a coordinated contraction of the ventricular muscle mass.

### SUMMARY

The techniques of this disclosure generally relate to a lead-in-lead system and methods for cardiac therapy. The lead-in-lead system allows the leads to be translatable or rotatable relative to one another to facilitate capturing desired parts of the patient's heart. In other words, the lead-in-lead system may provide a customized implantation solution for each patient.

In one aspect, the present disclosure provides a method that includes implanting a first implantable lead to implant a first electrode of the first implantable lead at a first implantation site in the coronary sinus or a coronary vein of a patient's heart. The method also includes advancing a second implantable lead having a second electrode guided by a distal portion of the first implantable lead to a second implantation site distal to the first electrode in a coronary vein of the patient's heart. The method further includes implanting the second electrode at the second implantation site.

In another aspect, the present disclosure provides a method that includes advancing a second implantable lead having a second electrode guided by a distal portion of a first implantable lead having a first electrode to a location in the coronary sinus or a coronary vein of a patient's heart. The method also includes implanting the first electrode at a first implantation site in the coronary sinus or a coronary vein of the patient's heart and puncturing through a vessel wall at the location in the coronary sinus or the coronary vein into the pericardial cavity of the patient's heart. The method further includes advancing the second implantable lead through the puncture in the vessel wall into the pericardial cavity and implanting the second electrode into the myocardium of the patient's heart from the pericardial cavity at a second implantation site distal to the first implantation site.

In another aspect, the present disclosure provides a method that includes implanting a first implantable lead to implant a first electrode of the first implantable lead at a first implantation site in the coronary sinus or a coronary vein of a patient's heart. The method also includes advancing a second implantable lead having a second electrode guided by a distal portion of the first implantable lead to a second implantation site distal to the first electrode in a left ventricular (LV) myocardium of the patient's heart. The method further includes implanting the second electrode at the second implantation site.

In another aspect, the present disclosure provides a system that includes a first implantable lead having a distal portion and a first electrode coupled to the distal portion of the first implantable lead. The first electrode is configured to be implanted at a first implantation site in the coronary sinus or a coronary vein of a patient's heart. The system also includes a second implantable lead having a distal portion and a second electrode coupled to the distal portion of the second implantable lead. The second electrode is configured to be implanted at a second implantation site distal to the first electrode in the patient's heart. The distal portion of the second implantable lead is guided by the distal portion of the first implantable lead to the first implantation site.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is an illustration of a patient's heart as one example of an environment for using the lead-in-lead systems and methods of the present disclosure.

FIGS. 2-3 are schematic illustrations of examples of lead-in-lead systems according to the present disclosure for use with, e.g., the patient's heart of FIG. 1.

FIGS. 4-5 are schematic illustrations of distal and proximal views, respectively, of the lead-in-lead system of FIG. 2.

FIGS. 6-7 are schematic illustrations of electrical connectors according to the present disclosure that may be used with, for example, the lead-in-lead systems of FIGS. 2-3, to couple leads to an IMD.

FIG. 8 is a flow diagram of one example of a method for delivering the lead-in-lead systems of FIGS. 2-3.

FIGS. 9-11 are schematic illustrations of various stages of delivering a lead-in-lead system for ventricle-from-atrium cardiac therapy according to the present disclosure for use with, e.g., the lead-in-lead systems of FIGS. 2-3.

FIG. 12 is a flow diagram of one example of a particular method for delivering a lead-in-lead system for ventricle-from-atrium cardiac therapy according to the present disclosure for use with, e.g., the lead-in-lead systems of FIGS. 2-3.

FIG. 13 is a schematic illustration of an environment for a lead-in-lead system, such as one of the lead-in-lead systems of FIGS. 2-3, for ventricle-from-atrium cardiac therapy with a defibrillator lead according to the present disclosure.

FIGS. 14-16 are schematic illustrations of various stages of delivering a lead-in-lead system for dual bundle-branch cardiac therapy according to the present disclosure for use with, e.g., the lead-in-lead systems of FIGS. 2-3.

FIG. 17 is a flow diagram of one example of a particular method for delivering a lead-in-lead system for dual bundle-branch cardiac therapy according to the present disclosure for use with, e.g., the lead-in-lead systems of FIGS. 2-3.

FIG. 18 is a schematic illustration of one example of a lead-in-lead system, such as one of the lead-in-lead systems of FIGS. 2-3, for dual bundle-branch cardiac therapy used with an atrial lead according to the present disclosure.

FIG. 19 is a schematic illustration of one example of a lead-in-lead system, such as one of the lead-in-lead systems of FIGS. 2-3, for dual bundle-branch cardiac therapy used with an atrial lead and a defibrillator lead according to the present disclosure.

FIGS. 20-22 are schematic illustrations of examples of lead-in-lead systems, such as the lead-in-lead systems of FIGS. 2-3, for left-side cardiac therapy in the coronary vasculature according to the present disclosure.

FIG. 23 is a schematic illustration of one example of an implantable medical device for use with, e.g., the lead-in-lead systems of FIG. 2 or FIG. 3.

### DETAILED DESCRIPTION

The present disclosure relates to a lead-in-lead system and methods for cardiac therapy. The lead-in-lead approach may provide efficient and effective delivery of electrodes to various locations in the patient's heart, including at the triangle of Koch for ventricle-from-atrium (VfA) therapy, at the right ventricular septal wall for dual bundle-branch pacing, and in the coronary vasculature for left side sensing and pacing. VfA therapy may relate to providing an electrode to capture the LV implanted through the AV septum, such as the RA-LV septal wall. The lead-in-lead system includes a first implantable lead and a second implantable lead. One of the leads may be a bipolar lead, while the other lead may be a unipolar lead. The leads may include electrodes that are passive to monitor electrical activity or active to provide pacing pulses. The lead-in-lead system allows the leads to be translatable or rotatable relative to one another to facilitate capturing desired parts of the patient's heart. In other words, the lead-in-lead system may provide a customized implantation solution for each patient.

As used in this specification and the appended claims, the term “or” is generally employed in its sense including “and/or” unless the content clearly dictates otherwise.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar.

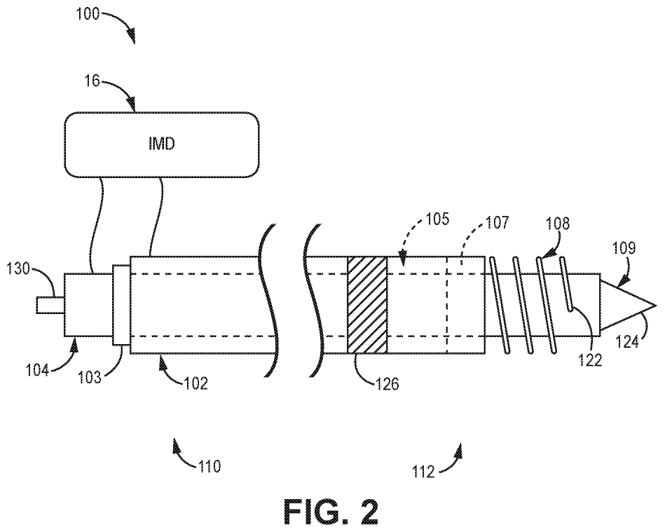

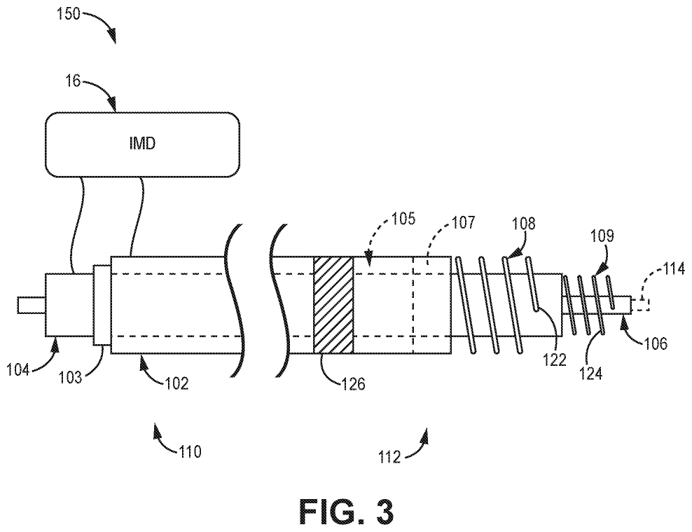

Various embodiments of this disclosure provide a lead-in-lead system for cardiac or other therapies. FIGS. 2 and 3 illustrate different examples of lead-in-lead systems. FIG. 2 shows a system 100 including a first implantable lead 102 and a second implantable lead 104, which are operably couplable an IMD 16, and further including a stylet 130. FIG. 3 shows a system 150 also including the first implantable lead 102, the second implantable lead 104, the IMD 16, and a guidewire 106 instead of the stylet 130. The system 100 and the system 150 may be similar in many aspects except where described differently herein.

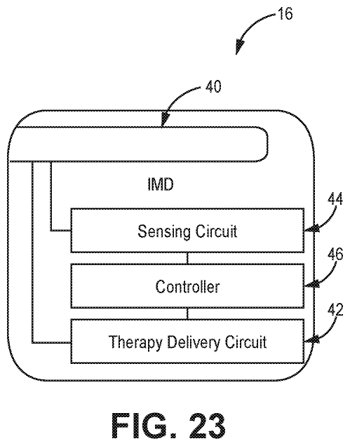

The IMD 16 may be any suitable device known to one of ordinary skill in the art having the benefit of this disclosure that can operably couple to one or more implantable leads and one or more electrodes to sense electrical activity or to deliver therapy. The IMD 16 may be, for example, an implantable pacemaker, cardioverter, and/or defibrillator that provides electrical signals to a patient's heart via electrodes coupled to one or more of leads. Further non-limiting examples of the IMD 16 include: a pacemaker with a medical lead, an ICD, an intracardiac device, a subcutaneous ICD (S-ICD), and a subcutaneous medical device (e.g., nerve stimulator, inserted monitoring device, etc.). One example of an IMD 16 is shown in FIG. 23.

Various implantation sites may be targeted using lead-in-lead systems. In one or more embodiments described herein, the systems are configured to be implanted in the triangle of Koch region in the atrioventricular wall of the patient's heart into the tissue structure between the right atrium and left ventricle of the patient's heart. In one or more embodiments described herein, the systems are configured to be implanted in the ventricular septal wall into the tissue structure is between the right ventricle and the left ventricle of the patient's heart. In some embodiments described herein, the systems are configured to be implanted in the coronary sinus or a coronary vein. Further, in some embodiments described herein, the system are configured to be implanted in the LV myocardium.

As can be seen in either FIG. 2 or FIG. 3, the first implantable lead 102 may be mechanically coupled to the second implantable lead 104 by a lead fixture element 103. In some embodiments, the first implantable lead 102 and the second implantable lead 104 may be formed separately. In such a configuration, the leads may be advanced one at a time. The second implantable lead 104 may be advanced through a lumen of the first implantable lead 102 to an implantation site, for example, after the first implantable lead has been fixed at the implantation site. In other embodiments, the first implantable lead 102 and the second implantable lead 104 may be formed into a single lead assembly or assembled lead configured to concurrently advance toward the implantation site.

The lead fixture element 103 may be described as a plug coupled between the leads. The lead fixture element 103 may facilitate translation or rotation of the leads relative to one another. In one or more embodiments described herein, the first implantable lead 102 is translatable along a longitudinal direction relative to the second implantable lead 104, which may facilitate proper positioning of the leads during implantation to sense electrical activity or to deliver cardiac therapy. The second implantable lead 104 may translated to extend, or be advanced, distally from an end of the first implantable lead 102 to penetrate further into the tissue structure or to extend distally further into a space than the first implantable lead.

As used herein, the “longitudinal” direction refers to a direction along or parallel to a direction between a proximal part and a distal part of an elongate member, such as an implantable lead.

The system 100 may define a proximal portion 110 and a distal portion 112, as well as a proximal end and a distal end. As illustrated, the double curve lines between the proximal portion 110 and the distal portion 112 indicate that the leads may be longer than shown. Each of the first implantable lead 102 and the second implantable lead 104 may also include a respective proximal portion and a respective distal portion, as well as a respective proximal end and a respective distal end.

The second implantable lead 104 may be at least partially received within the first implantable lead 102, for example, to guide the second implantable lead. In some embodiments, the first implantable lead 102 may include a lumen 105 sized to receive the second implantable lead 104. In some embodiments, an opening of lumen 105 may be larger than a cross-section of the second implantable lead 104. A sealing element 107 may be coupled between the implantable leads to seal the distal portion of the first implantable lead 102 from fluid ingress, for example, at or near the distal end. In other words, the sealing element 107 may prevent, or substantially prevent, fluid from entering inside the lumen 105 to the space between the implantable leads.

A guidewire or a stylet may be used to advance and guide the lead-in-lead system 100 to one or more implantation sites. The guidewire or stylet may be received at least partially into a lumen of the second implantable lead 104. As shown in FIG. 2, a stylet 130 may be at least partially received within the lumen of the second implantable lead 104 from the proximal end of the lead. A distal end of the stylet 130 may be retained within the distal portion of the second implantable lead 104, for example, proximal to a closed distal end of the second implantable lead. As shown in FIG. 3, a guidewire 106 may be at least partially received within the lumen of the second implantable lead 104 from the proximal end of the lead and may extend distally from the lead. In one or more embodiments described herein, the guidewire 106 may be used to pierce into or through a tissue structure of the patient's heart.

In other embodiments, the second implantable lead 104 may not include a lumen. For example, the guidewire 106 may extend through the lumen 105 of the first implantable lead 102, and the guidewire 106 may be exchanged with the second implantable lead 104 during an implantation process.

As used herein, a “tissue structure” refers to tissue forming any structures of the heart, such as a heart wall. The tissue structure may define a surface of the heart. Non-limiting examples of tissue structures include an atrioventricular septal wall (such as the RA-LV septum), a ventricular septal wall (such as the RV-LA septum), a wall forming the apex of the patient's heart, and a vessel wall (such as the vessel wall of the coronary sinus or a coronary vein).

The first implantable lead 102 may be used to guide the second implantable lead 104 to an implantation site or past a first implantation site to a second implantation site. In one or more embodiments described herein, the distal portion of the first implantable lead 102 may be used to guide the distal portion of the second implantable lead 104, for example, to the same implantation site. For example, in one or more embodiments described herein, the implantation site may be the triangle of Koch region in the atrioventricular septal wall of the patient's heart or the ventricular septal wall in the basal (e.g., high basal or high septal) region or apical (e.g., low septal or near the apex) region. Implantation in the triangle of Koch region of the atrioventricular septal wall may facilitate pacing of the His bundle or ventricular myocardium. Implantation in the basal region of the ventricular septal wall may facilitate pacing of the His bundle branches. Implantation in the apical region may facilitate pacing of Purkinje fibers.

In other embodiments, the distal portion of the first implantable lead 102 may be implanted at a first implantation site, and the distal portion of the second implantable lead 104 may be implanted distally at a second implantation site. For example, in one or more embodiments described herein, the first implantation site may be in the coronary sinus or a coronary vein of the patient's heart and the second implantation site may be distal to the first implantation site in the coronary vein or in the myocardium (e.g., left ventricular myocardium) of the patient's heart. In one or more embodiments described herein, the second implantation site may be in the myocardium through the epicardium of the patient's heart outside of the coronary sinus and the coronary veins. In other words, the second implantation site in the myocardium may be accessed using the pericardial cavity.

Implantation leads may be secured, or fixed, for implantation concurrently or independently. For example, the first implantation lead 102 may be fixed before, during, or after the second implantation lead 104 is fixed. In some embodiments, after one of the implantation leads is fixed for implantation, the other of the implantation leads may be secured. In one example, the second implantation lead 104 may be fixed for implantation after the first implantation lead 102 is fixed for implantation. In another example, the first implantation lead 102 may be fixed for implantation after the second implantation lead 104 is fixed for implantation.

The second implantable lead 104 may be freely rotatable relative to the first implantable lead 102, or vice versa, which may facilitate certain types of fixation for implantation. For example, a rotating motion may be used to secure the first implantable lead 102 or the second implantable lead 104 to an implantation site using a fixation element that responds to a rotating motion.

In one or more embodiments described herein, one or both implantation leads may include a fixation element used to secure the respective lead to a tissue structure at a selected implantation site. Non-limiting examples of fixation elements include a drill and a helix. In one or more embodiments, such as shown in FIG. 2, the system 100 includes a fixation element 108 of the first implantable lead 102 may include a helix structure, and a fixation element 109 of the second implantable lead 104 may include a drill structure. In one or more embodiments, such as shown in FIG. 3, the system 150 includes a fixation element 108 of the first implantable lead 102 may include a helix structure, and a fixation element 111 of the second implantable lead 104 may include a helix structure.

In general, any suitable type of drill or helix structure known to one of ordinary skill in the art having the benefit of this disclosure may be used for fixation elements. The fixation elements of the implantable leads may be configured to rotate in the same or opposite directions. For example, one fixation element may be configured to screw into a tissue structure when the respective lead is rotated clockwise and the other fixation element may be configured to screw into the tissue structure when the respective other lead is rotated clockwise (e.g., the same direction) or counterclockwise (e.g., the opposite direction). In addition, or as an alternative, to fixation elements, one or both implantation leads may include a canted lead structure to facilitate fixing the respective implantation lead in a vessel, such as the coronary sinus or a coronary vein.

In one or more embodiments described herein, a puncture element 114 may extend distally from the guidewire 106. The puncture element 114 may be formed integrally or separately from the guidewire 106. The puncture element 114 may have a drill structure that may be used to drill into a tissue structure at an implantation site. In one or more embodiments described herein, when using the puncture element 114, the fixation element 111 of the second implantable lead 104 may have a helix structure as shown in FIG. 3. In one or more embodiments described herein, when not using the guidewire 106, the fixation element 109 of the second implantable lead 104 may have a drill structure as shown in FIG. 2. In one or more other embodiments described herein, the guidewire 106 having the puncture element 114 may be used with the fixation element 109 having a drill structure. For example, the guidewire 106 may extend through a lumen of the fixation element 109 and extend distally from the fixation element 109. In other words, the guidewire 106 may be used with the system 100 of FIG. 2 instead of the stylet 130.

The puncture element 114 may be used to provide a micropuncture through a tissue structure, such as a vessel wall, of the patient's heart. For example, the micropuncture into the pericardial cavity of the patient's heart may be formed at a location along the coronary sinus or a coronary vein. In other words, the micropuncture may be formed inferior to the coronal oblique transseptal plane of the patient's heart. In some embodiments, the puncture element 114 includes a microneedle configured to provide the micropuncture in the coronary sinus or coronary vein.

As used herein, the term “micropuncture” refers to a puncture through a wall that has a largest dimension, for example, along a major axis or a diameter, of less than about 1 millimeter or on the order of about 100 micrometers, about 10 micrometers, or about 1 micrometer.

When forming a puncture in a vessel wall, a structure of the first implantable lead 102 may be configured to seal the puncture after implantation. In some embodiments, the structure may include a lead body or sheath of the implantable lead. For example, the first implantable lead 102 may be fixed against the vessel wall toward the pericardial cavity. The vessel wall may be sealed by the active fixation, and the second implantable lead 104 may extend radially from the first implantable lead 102, through the vessel wall, and into the pericardial cavity. In some embodiments, the structure may include the sealing element 107.

As used herein, the phrase “to seal the puncture” refers to partial sealing, complete sealing, or almost complete sealing of the puncture between a vessel and a space of the patient's heart. For example, the sealing of the puncture may be sufficient to prevent major tamponade (e.g., resulting from fluid buildup in the pericardial cavity).

One or both of the implantable leads includes one or more electrodes. In some embodiments described herein, the first implantable lead 102 includes a first electrode 122 coupled to the first implantable lead, and the second implantable lead 104 includes a second electrode 124 coupled to the second implantable lead. Each fixation element may be formed integrally or separately from the respective electrode. In the illustrated embodiments shown in FIG. 2 and FIG. 3, the electrodes are integrally formed with their respective fixation elements. The integrally formed electrodes are configured to pierce into the tissue structure of the patient's heart. In one or more embodiments described herein, the integrally formed electrodes are formed at a distal portion or end of the respective fixation element.

The first electrode 122 or the second electrode 124 may disposed on the distal portion or at the distal end of the respective implantable lead. In one or more embodiments described herein, the first electrode 122 or the second electrode 124 may be disposed proximal to a distal end of the respective implantable lead, for example, when a structure of the respective implantable lead is used to seal a puncture in a tissue structure, such as a vessel wall.

The first electrode 122 and the second electrode 124 may be described as cathode electrodes (or simply cathodes) for pacing or sensing. For example, the electrodes may provide various pacing vectors for different cardiac therapies, such as CRT. The first implantable lead 102 or the second implantable lead 104 may include a return electrode 126, which may be described as a common anode electrode (or simply an anode). In the illustrated embodiments of FIG. 2 and FIG. 3, the return electrode 126 is coupled to the first implantable lead 102 and is shown as a ring electrode. The return electrode 126 may be proximal or distal to the cathode electrodes. The return electrode 126 may be proximal to the first electrode 122 and proximal to the second electrode 124. The implantable lead having two electrodes (e.g., cathode and anode electrodes) may be described as a bipolar lead.

In one or more embodiments described herein, the second implantable lead 104 includes multiple second electrodes 124 (e.g., a plurality of second electrodes), which may be used to provide multiple sensing or pacing sites along the lead. The first electrode 122 and the return electrode 126 may be proximal to one or more of the multiple second electrodes 124.

In one or more embodiments described herein, the first electrode 122 is configured to be implanted on or in a tissue structure of a patient's heart at the implantation site, and the second electrode 124 is configured to be implanted at the same implantation site distal to the first electrode within the tissue structure of the patient's heart. After one of the electrodes is implanted, the other electrode may be translatable relative to the implanted electrode. For example, the second electrode 124 may be translatable relative to the first electrode 122, even after the first electrode is implanted, to allow the second electrode to be implanted at various depths within the tissue structure of the patient's heart. The translatable coupling of the first implantation lead 102 and the second implantation lead 104 may facilitate the translatable relationship between the electrodes.

In one or more embodiments described herein, the first electrode 122 is configured to be implanted at a first implantation site in the coronary sinus or in a coronary vein of the patient's heart, and the second electrode 124 is configured to be implanted at a second implantation site distal to the first electrode in the patient's heart. For example, the second electrode 124 may be implanted in a coronary vein.

One or more of the electrodes may be configured to sense electrical activity or to provide pacing pulses (e.g., in delivering cardiac therapy). In one or more embodiments described herein, both the first electrode 122 and the second electrode 124 are configured to provide pacing pulses. One or both electrodes may also be configured to sense electrical activity. In one or more embodiments described herein, the first electrode 122 is configured to only sense electrical activity, and the second electrode 124 is configured to provide pacing pulses. The second electrode 124 may also be configured to sense electrical activity. In some embodiments, a plurality of second electrodes 124 may be configured to deliver cardiac therapy to or sense electrical activity of the left ventricle.

In general, one or more of the electrodes may be configured to deliver electrical pulses to test one or more locations of the respective electrode in the patient's heart. In one or more embodiments described herein, one or more depths of the respective electrode in the tissue structure of the patient's heart may be tested. The test electrical pulses may facilitate identifying an appropriate depth or an appropriate surface location on a tissue structure for the electrode to capture the desired sensing or pacing. In one or more embodiments described herein, the first electrode 122 or the second electrode 124 may be tested at variable depths or surface locations before being implanted at a selected depth. In one example, an implantation depth or surface location of the first electrode 122 may be tested to determine whether the RBB is captured, and the implantation depth of the second electrode 124 may be tested to determine whether the LBB is captured. In another example, a surface location of the first electrode 122 may be tested to determine whether the RA myocardium is captured, and the implantation depth of the second electrode 124 may be tested to determine whether the LV myocardium or His bundle is captured.

In one or more embodiments described herein, one or more locations of the respective electrode in the patient's heart may be tested. In one example, the first electrode 122 may be tested to determine whether the LA is captured, and the second electrode 124 may be tested to determine whether the LV is captured. The electrodes may be advanced through a vessel, such as the coronary sinus or a coronary vein, of the patient's heart. One or more locations of the first electrode 122 in and along the vessel may be tested, for example, to capture the LA. Additionally, or alternatively, one or more locations of the second electrode 124 in the vessel or in the myocardium along the vessel may be tested, for example, to capture the LV. Further, one or more of the electrodes may be advanced into a space of the patient's heart, such as the pericardial cavity. One or more locations of the second electrode 124 in the myocardium along the pericardial cavity may be tested, for example, to capture the LV.

Although both electrodes may be implanted at the same implantation site, different parts of the patient's heart may be captured by advancing the electrodes to different depths. In one or more embodiments described herein, the first electrode 122 is implantable in the RA of the patient's heart to deliver cardiac therapy to or sense electrical activity of the RA of the patient's heart, and the second electrode 124 is implantable from the triangle of Koch region of the RA of the patient's heart to deliver cardiac therapy to or sense electrical activity of the LV in the basal region, septal region, or basal-septal region of the left ventricular myocardium of the patient's heart for VfA cardiac therapy.

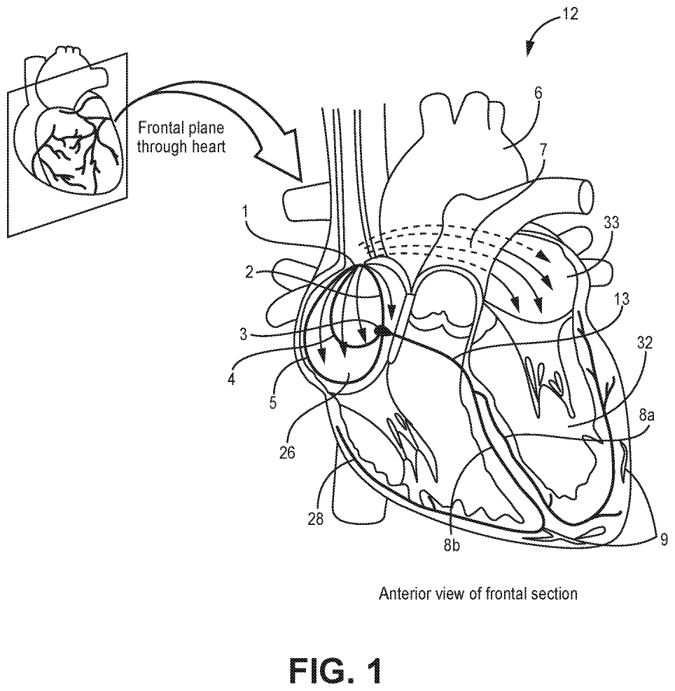

In one or more embodiments described herein, the first electrode 122 is implantable closer to a first bundle branch of the cardiac conduction system of the patient's heart than the second electrode 124, and the second electrode is implantable closer to a second bundle branch of the cardiac conduction system of the patient's heart than the first electrode. For example, the first electrode 122 may be implanted closer to the RBB 8b (FIG. 1), and the second electrode 124 may be implanted closer to the LBB 8a (FIG. 1). The implantable leads may be implanted from the RV into the ventricular septal wall toward the LV, which may facilitate placing the first electrode 122 in close proximity to the RBB 8b and placing the second electrode 124 in close proximity to the LBB 8a. The electrodes may be used to deliver dual-bundle branch cardiac therapy (e.g., using pacing pulses) to or sense electrical activity (e.g., electrical sensing) of the RBB 8b and the LBB 8a.

One or both implantable leads may include a sheath, or lead body, and one or more conductors extending inside the sheath. In one or more embodiments described herein, the systems may include at least three electrodes, including two cathode electrodes and one anode electrode. For example, one of the leads may have two electrodes and the other lead may have one electrode. Each electrode may be operably coupled to a distal portion of a conductor that extends through the sheath of the implantable lead. A proximal portion of the conductor may be operably coupled to one or more electrical connectors at the proximal portion of the lead.

Various types of electrical connectors may be used to provide an operative connection between a medical device, which may be implantable, and one or more conductors. In one or more embodiments described herein, one example of an electrical connector has a bifurcated proximal end. Each branch may include or be described as an IS connector. In some embodiments, a further example of an electrical connector has two separate IS connectors. In one or more embodiments described herein, another example of an electrical connector has an inner conductor and two outer conductors. The inner conductor may be operably coupled to the second electrode 124, and the two outer conductors may be operably coupled to the first electrode 122 and the return electrode 126. In some embodiments, such an electrical connector may be described as an IS-4 connector.

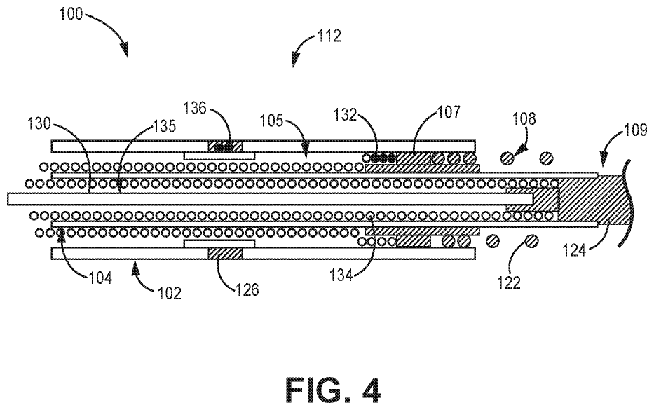

FIG. 4 illustrates a cross-sectional view of one example of the distal portion 112 of the lead-in-lead system 100 that includes various conductors operably coupled to the electrodes. Any suitable conductors known to one of ordinary skill in the art having the benefit of this disclosure may be used for extending through the implantable leads of the system 100.

The second implantable lead 104 may extend through the lumen 105 formed by the first implantable lead 102. The second implantable lead 104 may also define a lumen 135, and a stylet 130 may extend at least partially through the lumen to facilitate control of advancement and direction of the distal portion 112 of the system 100 during an implantation process.

The first electrode 122 is integrally formed with the fixation element 108 and coupled to the first implantable lead 102. The first electrode 122 is operably coupled to a first conductor 132 extending through the first implantable lead 102. The first conductor 132 may be disposed in the lumen 105 of the first implantable lead 102. The electrode of the conductor may extend through the sealing element 107 to establish an electrical connection between the first electrode 122 and the first conductor 132.

Also, as illustrated, the second electrode 124 is integrally formed with the fixation element 109 and coupled to the second implantable lead 104. The second electrode 124 is operably coupled to a second conductor 134 extending through the second implantable lead 104. The second conductor 134 may be disposed in the lumen 135 of the second implantable lead 104. Another conductor also may also extend through the first implantable lead 102. As illustrated, the return electrode 126 is operably coupled to a third conductor 136 extending through the lumen 105 of the first implantable lead 102. Any suitable conductor type known to one of ordinary skill in the art having the benefit of this disclosure may be used. In the illustrated embodiment, each of the conductors are coil conductors, which may facilitate flexibility of the respective lead.

In some embodiments, the distal portion of the second implantable lead 104 may include a monolithic controlled release device (MCRD). The MCRD may be positioned proximal to the second electrode 124.

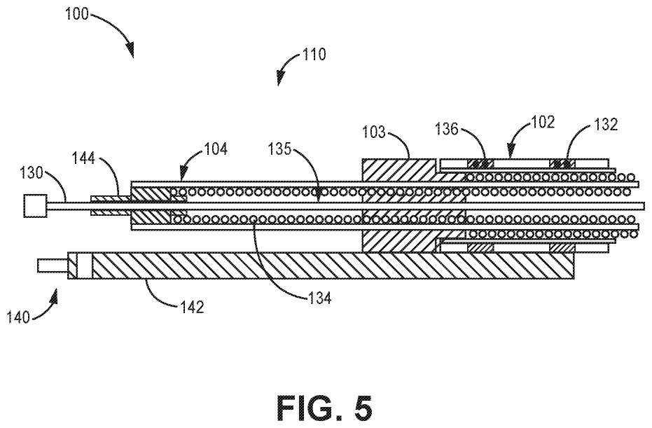

FIG. 5 illustrates a cross-sectional view of one example of the proximal portion 110 of the lead-in-lead system 100 that includes an electrical connector operably coupled to the conductors. As illustrated, an electrical connector 140 includes a first IS connector 142 and a second IS connector 144. The first conductor 132 and the third conductor 136 are operably coupled to the first IS connector 142, for example, using coil joints. The first conductor 132 may couple to a proximal contact at a proximal end or tip of the first IS connector 142, and the third conductor 136 may couple to a proximal ring contact distal to the proximal end or tip. The second conductor 134 is operably coupled to the second IS connector 144, which has at least one proximal contact. The proximal contacts of the IS connectors may be used to operably couple to the IMD 16 of FIG. 2 or FIG. 3.

In the illustrated embodiment, the first implantable lead 102 and the second implantable lead 104 are coupled by the lead fixture element 103. The stylet 130 extends through the lumen 135 of the second implantable lead 104 and through the lead fixture element 103 and may be proximally coupled to a stylet driver.

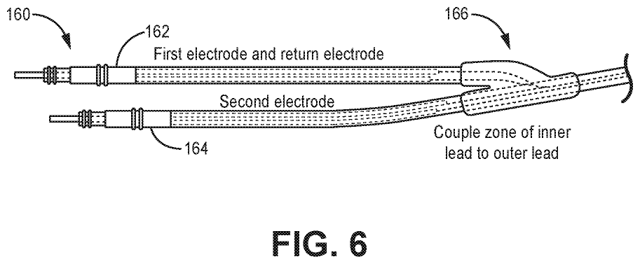

FIG. 6 illustrates one example of a proximal end of a lead-in-lead system having a bifurcated electrical connector 160. The electrical connector 160 has a bifurcated proximal end. Two electrodes, such as the first electrode 122 and the return electrode 126 of FIG. 2 or FIG. 3, may be operably coupled to conductors extending through one branch 162 of the bifurcated proximal end and another electrode, such as the second electrode 124 of FIG. 2 or FIG. 3, may be operably coupled to a conductor extending through the other branch 164. Each branch may be described as an IS connector. The electrical connector 160 may include a coupling zone 166 where the electrical connector 160 may interface with the implantable leads. The electrical connector 160 may have the same or similar proximal contacts as the electrical connector 140 for coupling to the IMD 16.

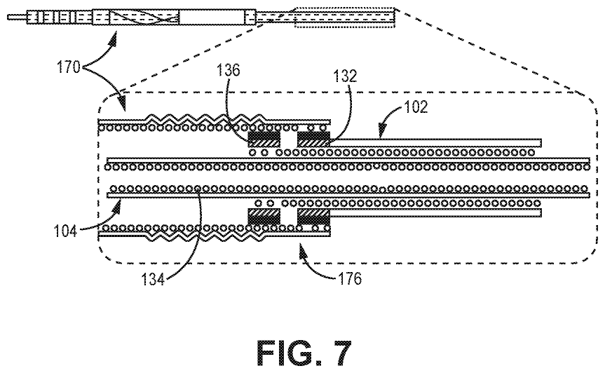

FIG. 7 illustrates a cross-sectional view of one example of a coupling zone 176 of an electrical connector 170 usable in a lead-in-lead system. The coupling zone 176 may be coupled to the first implantable lead 102 to operably couple to the first conductor 132 and the third conductor 136. The second conductor 134 of the second implantable lead 104 may extend through the coupling zone 176 and operably couple to a proximal contact at a proximal end or tip of the electrical connector 170. The first conductor 132 and the third conductor 136 may each operably couple to a proximal ring contact distal to the proximal end or tip.

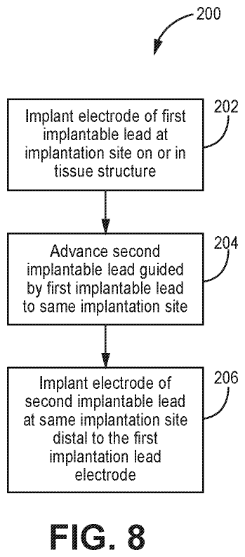

Various embodiments of this disclosure provide a method of delivering lead-in-lead systems for cardiac therapy to target various areas within the patient's heart. FIG. 8 illustrates one example of a method 200 for delivering a lead-in-lead system, which includes implanting a first electrode of a first implantable lead at an implantation site on or in a tissue structure of a patient's heart 202. The method 200 may also include advancing a second implantable lead having a second electrode guided by a distal portion of the first implantable lead to the same implantation site 204. The method 200 may further include implanting the second electrode at the same implantation site distal to the first implantation lead electrode 206.

The first implantable lead and the second implantable lead may be advanced concurrently toward the implantation site. In some embodiments, implanting the second electrode includes translating the second electrode relative to the first electrode after the first electrode has been implanted to allow the second electrode to be implanted at various selectable depths within the tissue structure of the patient's heart. Implanting the second electrode may also include delivering electrical pulses to test one or more depths of the first or second electrode in the tissue structure of the patient's heart.

In some embodiments, implanting the electrode of the first implantable lead may include advancing a guidewire to the implantation site. The guidewire may pierce or puncture the surface of the tissue structure. The first implantable lead may be guided over the guidewire to the implantation site. When the first implantable lead has been appropriately implanted, the guidewire may optionally be removed. The second implantable lead may be guided by a lumen of the first implantable lead or be guided over the guidewire to the implantation site. The second implantable lead may advance (e.g., drill or screw) distally into the tissue structure at the implantation site.

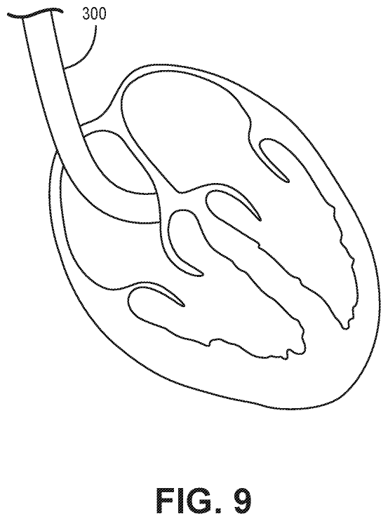

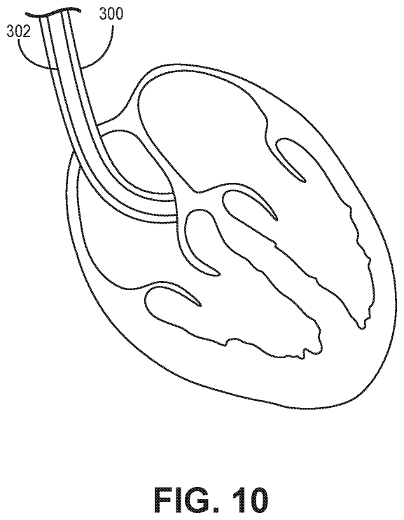

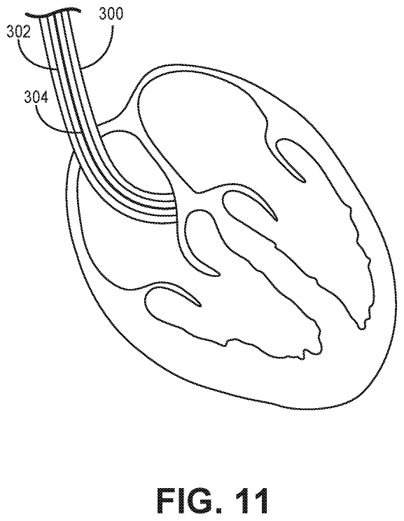

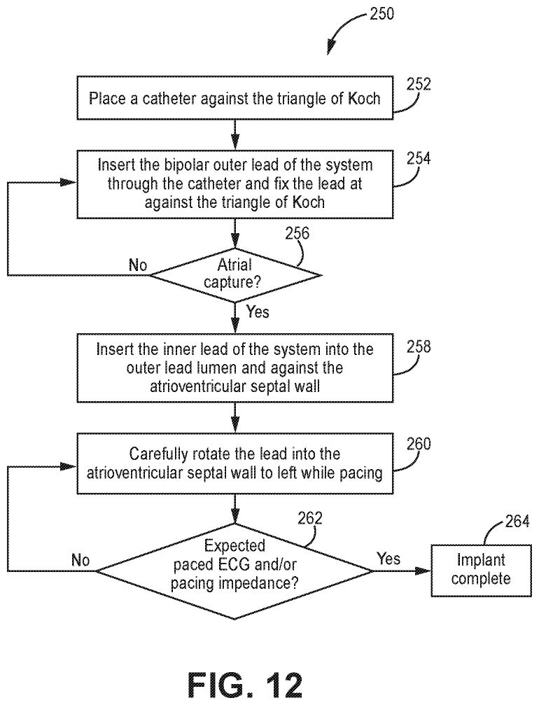

FIG. 9, FIG. 10, and FIG. 11 schematically illustrate various stages of one example of carrying out the method 200, in particular, to deliver a lead-in-lead system, such as lead-in-lead system 100 (FIG. 2) or lead-in-lead system 150 (FIG. 3), for VfA cardiac therapy. In FIG. 9, a deflectable catheter 300 is advanced to and placed against the triangle of Koch in the patient's heart. In FIG. 10, a bipolar outer lead 302 (e.g., first implantable lead) of the system is implanted through the catheter 300 and is fixated at the triangle of Koch next to coronary sinus ostium. In FIG. 11, once the bipolar outer lead 302 of the system is fixed at triangle of Koch next to coronary sinus ostium, the inner lead 304 (e.g., second implantable lead) of the system is implanted though the outer lead lumen and fixated into the atrioventricular septal wall by rotating the inner lead body. FIG. 12 is a flowchart illustrating one example of a particular method 250 for carrying out the method 200, in particular, to deliver a lead-in-lead system for VfA cardiac therapy. The method 250 may include placing a catheter against the triangle of Koch 252 (see FIG. 9). The method 250 may also include inserting the bipolar outer lead of the system through the catheter and fixing the lead against the triangle of Koch 254 (see FIG. 10). The method 250 may determine whether the bipolar outer lead captures the atrium 256, in particular, the right atrium. If the atrium is not captured, the method 250 may return to inserting the bipolar lead 254 into a new location in the triangle of Koch. If the atrium is captured, the method 250 may include inserting the inner lead of the system into the outer lead lumen and placing the inner lead against the atrioventricular septal wall 258 (see FIG. 11). The method 250 may also include carefully rotating the inner lead into the atrioventricular septal wall toward the left side of the patient's heart while performing test pacing 260 (see FIG. 11).

The method 250 may further determine whether an expected paced ECG or pacing impedance is observed during the inner lead rotating process 262. If the expected ECG or pacing impedance is observed, the method 250 may determine that implantation is complete 264. For example, the leads may then be coupled to an IMD. Otherwise, the method 250 may continue to rotate the inner lead while test pacing 260.

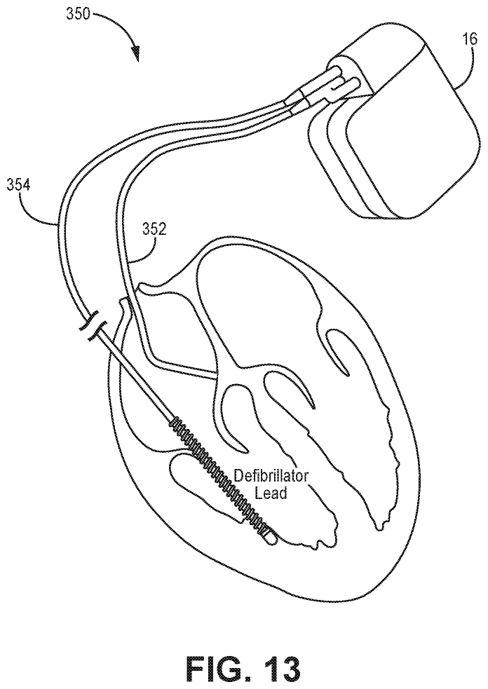

FIG. 13 illustrates a system 350 that may utilize a lead-in-lead system 352, which may be lead-in-lead system 100 (FIG. 2) or lead-in-lead system 150 (FIG. 3), and a defibrillator lead 354 both coupled to the IMD 16 to provide cardiac therapy that may include VfA pacing (e.g., DDDR-type pacing) and defibrillation. The defibrillator lead 354 may be implanted, for example, in the RV of the patient's heart.

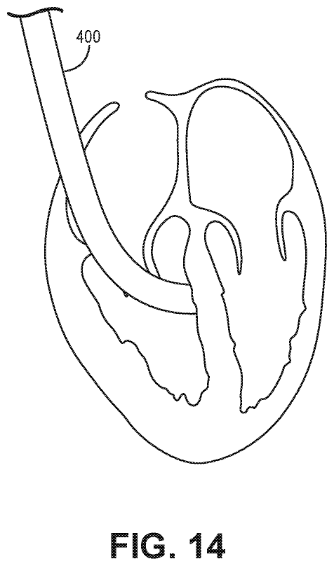

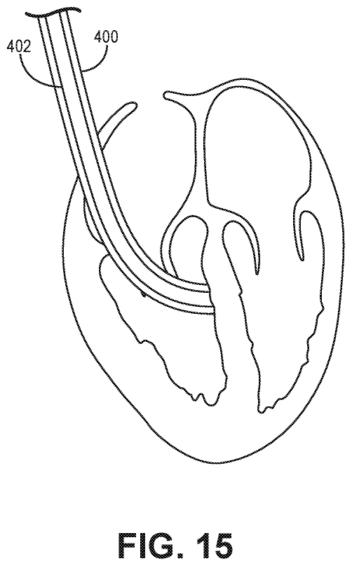

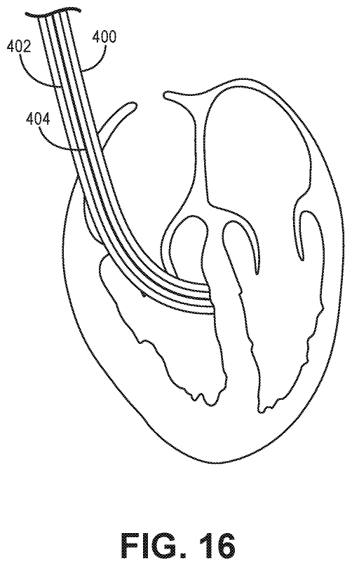

FIG. 14, FIG. 15, and FIG. 16 schematically illustrate various stages of one example of carrying out method 200, in particular, to deliver a lead-in-lead system, such as lead-in-lead system 100 (FIG. 2) or lead-in-lead system 150 (FIG. 3), for bi-cardiac conduction branch pacing or, in other words, dual bundle-branch cardiac therapy. In FIG. 14, a deflectable catheter 400 is advanced to and placed against the RV high septal wall under the antero-septal tricuspid commissure, for example, about 1 to 3 centimeters (cm) below. In FIG. 15, a bipolar outer lead 402 (e.g., first implantable lead) of the system is implanted through the catheter 400 and is fixated at the right ventricular septal wall under the antero-septal tricuspid commissure, for example, about 1 to 3 cm below. In FIG. 16, once the bipolar outer lead 402 of the system is fixed at the right ventricular septal wall, the inner lead 404 (e.g., second implantable lead) of the system is implanted though the outer lead lumen and fixated into septal wall to left high septal wall (e.g., at the bundle branches) by rotating the inner lead body.

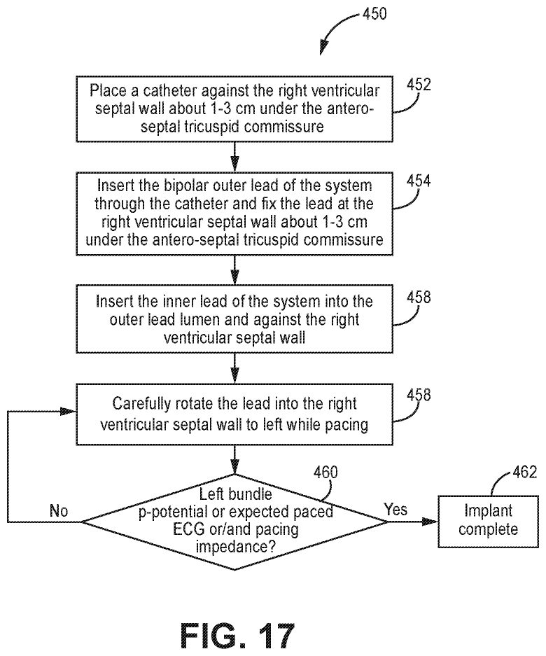

FIG. 17 is a flowchart illustrating on example of a particular method 450 for carrying out the method 200, in particular, to deliver a lead-in-lead system, such as lead-in-lead system 100 (FIG. 2) or lead-in-lead system 150 (FIG. 3), for dual bundle-branch cardiac therapy. The method 450 may include placing the catheter against the right ventricular septal wall under the antero-septal tricuspid commissure 452 (see FIG. 14), for example, about 1 to 3 cm below. The method 450 may also include inserting the bipolar outer lead of the system through the catheter and fixing the bipolar outer lead at the right ventricular septal wall under the antero-septal tricuspid commissure 454 (see FIG. 15), for example, about 1 to 3 cm below.

The method 450 may further include inserting the inner lead of the system into the outer lead lumen and placing the inner lead against the right ventricular septal wall 456 (see FIG. 16). The method 450 may include carefully rotating the inner lead into the right ventricular septal wall toward the left side of the patient's heart while performing test pacing 458 (see FIG. 16).

The method 450 may include determining whether a left bundle Purkinje potential from an EGM using the second electrode or an expected pacing impedance is observed 460. If the left bundle Purkinje potential or pacing impedance is observed, the method 450 may determine that implantation is complete 462. For example, the leads may then be coupled to an IMD. Otherwise, the method 450 may continue to rotate the inner lead while test pacing 458.

Optionally, the method 450 also include rotating the bipolar outer lead and performing test pacing until a right bundle Purkinje-potential, expected EGM, or pacing impedance is observed.

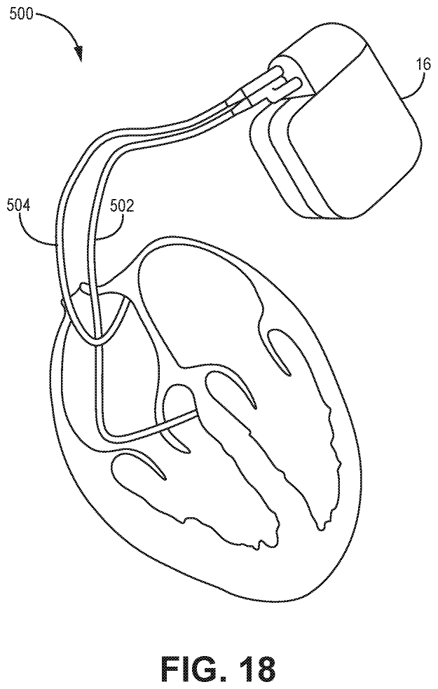

FIG. 18 illustrates a system 500 that may utilize a lead-in-lead system 502, which may be lead-in-lead system 100 (FIG. 2) or lead-in-lead system 150 (FIG. 3), and an atrial lead 504 both coupled to the IMD 16 to provide cardiac therapy that may include dual bundle-branch pacing (e.g., DDDR-type pacing). The atrial lead 504 may be planted in the RA of the patient's heart.

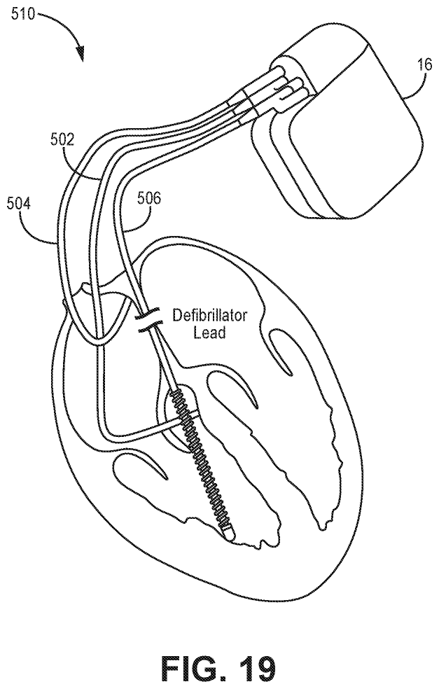

FIG. 19 illustrates a system 510 that may utilize a lead-in-lead system 502, which may be lead-in-lead system 100 (FIG. 2) or lead-in-lead system 150 (FIG. 3), an atrial lead 504, and a defibrillator lead 506 each coupled to the IMD 16 to provide cardiac therapy that may include dual bundle-branch pacing (e.g., DDDR-type pacing) and defibrillation. The defibrillator lead 506 may be implanted, for example, in the RV of the patient's heart.

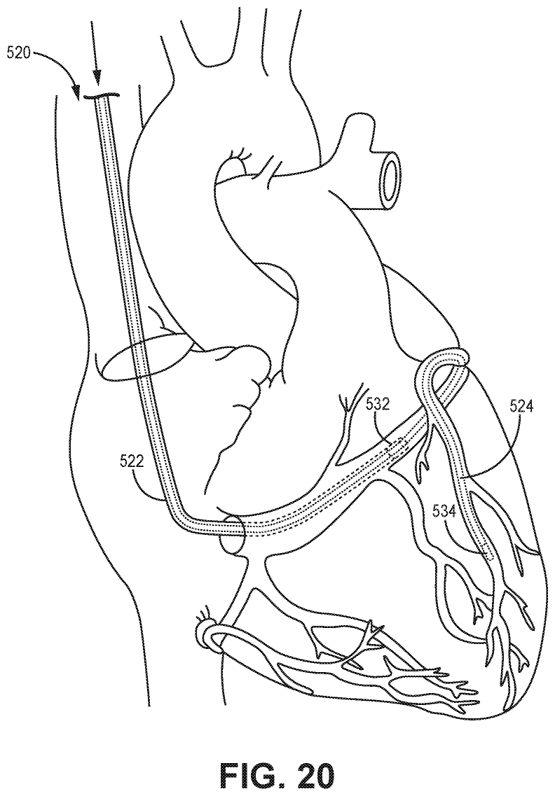

Various lead-in-lead systems may also be used to access the coronary vasculature, such as the coronary sinus and coronary veins, for left-side cardiac therapy. FIG. 20 shows one example of a system 520, which may be similar to or the same as lead-in-lead system 100 (FIG. 2) or lead-in-lead system 150 (FIG. 3), for left-side cardiac therapy. The system 520 includes a first implantable lead 522 and a second implantable lead 524. A first electrode 532 may be coupled to a distal portion of the first implantable lead 522 (e.g., outer lead) and implanted in a coronary vein (e.g., great cardiac vein). The second implantable lead 524 (e.g., inner lead) may extend distally from the first implantable lead 522. A second electrode 534 may be coupled to a distal portion of the second implantable lead 524. The second electrode 534 is positioned distally from the first electrode 532 (e.g., further along the great cardiac vein). The first electrode 532 may capture the LA of the patient's heart, and the second electrode 534 may capture the LV of the patient's heart.

The system 520 may be implanted using any suitable technique. One example of an implantation method may include implanting the first implantable lead 522 to implant the first electrode 532 at a first implantation site in the coronary sinus or a coronary vein of a patient's heart. The method may also include advancing the second implantable lead 524 having the second electrode 534 guided by a distal portion of the first implantable lead 522 to a second implantation site distal to the first electrode 532 in a coronary vein of the patient's heart. The method may further include implanting the second electrode 534 at the second implantation site.

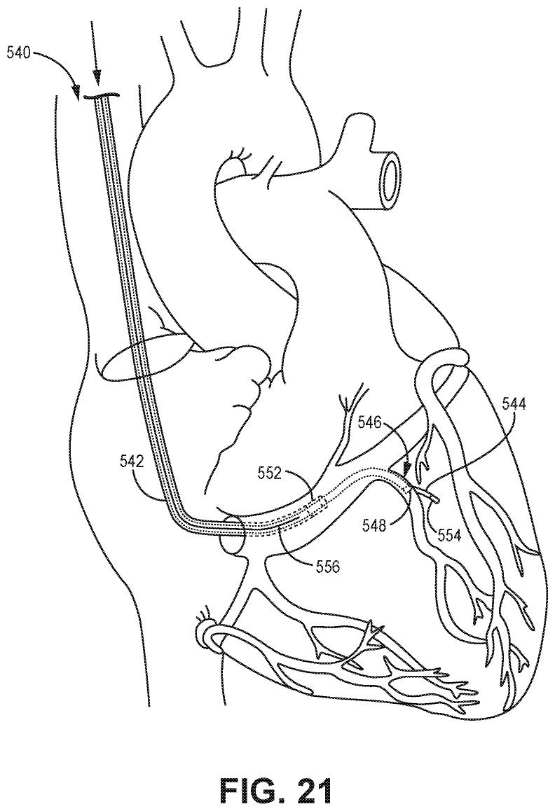

FIG. 21 shows one example of a system 540, which may be similar to or the same as lead-in-lead system 100 (FIG. 2) or lead-in-lead system 150 (FIG. 3), for left-side cardiac therapy. The system 540 includes a first implantable lead 542 and a second implantable lead 544. A first electrode 552 may be coupled proximal to a distal portion 546 of the first implantable lead 542 (e.g., outer lead) and implanted in the coronary sinus. The second implantable lead 544 (e.g., inner lead) may extend distally from the first implantable lead 542 and through a puncture 548, such as a micropuncture, in a coronary vessel wall (e.g., the wall of the middle cardiac vein). A second electrode 554 may be coupled to a distal portion of the second implantable lead 544 and may extend into the pericardial cavity of the patient's heart and be implanted into the myocardium, such as the LV myocardium, through the epicardium. The first electrode 552 may capture the LA of the patient's heart, and the second electrode 554 may capture the LV of the patient's heart.

In particular, the second implantable lead 544 may extend through the puncture 548 from the vessel into the pericardial cavity, which extends between the epicardium and the pericardium of the patient's heart. The distal end of the second implantable lead 544 may be positioned at various locations within the pericardial cavity to access different implantation sites in the myocardium, such as the LV myocardium, through the epicardium that are outside of the coronary vasculature.

A guidewire 556 may be used to form the puncture in the vessel wall and thereafter retracted from the system 540. A structure of the first implantable lead 542, such as the distal portion 546, may be advanced to the puncture 548 and positioned adjacent to the puncture to seal the puncture.

The system 540 may be implanted using any suitable technique. One example of an implantation method may include advancing the second electrode 554 of the second implantable lead 544 guided by the distal portion 546 of the first implantable lead 542 to a location in the coronary sinus or a coronary vein of a patient's heart. The method may also include implanting the first electrode 552 at a first implantation site in the coronary sinus or a coronary vein of the patient's heart. The method may also include puncturing through a vessel wall at the location in the coronary sinus or the coronary vein into the pericardial cavity of the patient's heart, for example, using the guidewire 556. The method may also include advancing the second implantable lead 544 through the puncture 548 in the vessel wall into the pericardial cavity. Further, the method may include implanting the second electrode 554 into the epicardium and myocardium of the patient's heart from the pericardial cavity at a second implantation site distal to the first implantation site. The second implantation site may be in the myocardium of the patient's heart outside of the coronary sinus or the coronary veins. In some embodiments, the first electrode 552 is implanted after the second electrode 554 is implanted, which may facilitate sealing of the puncture 548 by the first implantable lead 542.

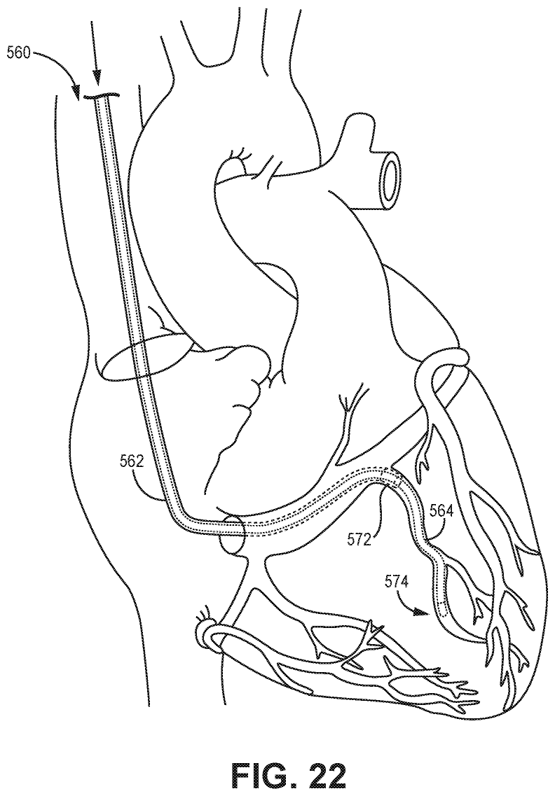

FIG. 22 shows one example of a system 560, which may be similar to or the same as lead-in-lead system 100 (FIG. 2) or lead-in-lead system 150 (FIG. 3), for left-side cardiac therapy. The system 560 includes a first implantable lead 562 and a second implantable lead 564. A first electrode 572 may be coupled to a distal portion of the first implantable lead 562 (e.g., outer lead) and implanted in the middle cardiac vein (e.g., a coronary vein). The second implantable lead 564 (e.g., inner lead) may extend distally from the first implantable lead 562. A second electrode 574 may be coupled to a distal portion of the second implantable lead 564 and may extend into the myocardium of the patient's heart, such as the LV myocardium. The first electrode 572 may capture the LA of the patient's heart, and the second electrode 574 may capture the LV of the patient's heart.

FIG. 23 shows one example of the IMD 16 including a connector receptacle 40 configured to receive a lead or lead connector from a lead-in-lead system, such as lead-in-lead system 100 (FIG. 2) or lead-in-lead system 150 (FIG. 3), a therapy delivery circuit 42 operably coupled to the connector receptacle, a sensing circuit 44 operably coupled to the connector receptacle, and a controller 46 operably coupled to the therapy delivery circuit and the sensing circuit.

The therapy delivery circuit 42 is configured to deliver cardiac therapy to the patient's heart through one or more operably connected electrodes, for example, electrically connected via the connector receptacle 40. The sensing circuit 44 is configured to sense electrical activity of the patient's heart using one or more operably connected electrodes, for example electrically connected via the connector receptacle 40. The electrodes operably coupled to the sensing circuit 44 may or may not include some or all of the electrodes that are also operably coupled to the therapy delivery circuit 42. The sensing circuit 44 may monitor electrical activity of the patient's heart, for example, using electrical signals, such as electrocardiogram (ECG) signals or electrogram (EGM) signals.

The controller 56 may have processing circuitry operably coupled to the therapy delivery circuit 42 and the sensing circuit 44. The controller 46 may be used to carry out various functionality of the IMD 16 coupled to the lead-in-lead systems described herein, such as lead-in-lead system 100 (FIG. 2) or lead-in-lead system 150 (FIG. 3).

Processing circuitry may include any one or more of a microprocessor, a controller, a digital signal processor (DSP), an application specific integrated circuit (ASIC), a field-programmable gate array (FPGA), or equivalent discrete or integrated logic circuitry. In some examples, processing circuitry may include multiple components, such as any combination of one or more microprocessors, one or more controllers, one or more DSPs, one or more ASICs, or one or more FPGAs, as well as other discrete or integrated logic circuitry. The functions attributed to the processing circuitry of the controller herein may be embodied as software, firmware, hardware or any combination thereof. The controller may control the therapy delivery circuit to deliver stimulation therapy to the patient's heart according to a selected one or more of therapy programs, which may be stored in a memory. Specifically, the controller may control the therapy delivery circuit to deliver electrical pulses with amplitudes, pulse widths, frequency, or electrode polarities specified by the selected one or more therapy programs.

The controller 46 may include memory. Non-limiting examples of memory may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Illustrative Aspects of Lead-in-Lead Systems and Methods

While the present disclosure is not so limited, an appreciation of various aspects of the disclosure will be gained through a discussion of the specific illustrative lead-in-lead system or methods thereof are provided below. Various modifications of the examples and illustrative aspects, as well as additional aspects or features of the disclosure, will become apparent herein.

In illustrative aspect A1, a method includes implanting a first implantable lead to implant a first electrode of the first implantable lead at a first implantation site in the coronary sinus or a coronary vein of a patient's heart. The method also includes advancing a second implantable lead having a second electrode guided by a distal portion of the first implantable lead to a second implantation site distal to the first electrode in a coronary vein of the patient's heart. The method further includes implanting the second electrode at the second implantation site.

In illustrative aspect A2, a method includes the method of any A aspect, wherein the first electrode is implanted in the coronary sinus or a coronary vein of the patient's heart and the second electrode is implanted in a coronary vein of the patient's heart distal to the first electrode.

In illustrative aspect A3, a method includes the method of any A aspect, further including: delivering cardiac therapy to or sensing electrical activity of the left atrium (LA); and delivering cardiac therapy to or sensing electrical activity of the left ventricle (LV).

In illustrative aspect B1, a method includes advancing a second implantable lead having a second electrode guided by a distal portion of a first implantable lead having a first electrode to a location in the coronary sinus or a coronary vein of a patient's heart. The method also includes implanting the first electrode at a first implantation site in the coronary sinus or a coronary vein of the patient's heart and puncturing through a vessel wall at the location in the coronary sinus or the coronary vein into the pericardial cavity of the patient's heart. The method further includes advancing the second implantable lead through the puncture in the vessel wall into the pericardial cavity and implanting the second electrode into the myocardium of the patient's heart from the pericardial cavity at a second implantation site distal to the first implantation site.

In illustrative aspect B2, a method includes the method of any B aspect, wherein the first electrode is implanted in the coronary sinus or a coronary vein of the patient's heart and the second electrode is implanted into the myocardium of the patient's heart outside of the coronary sinus or the coronary veins distal to the first electrode.

In illustrative aspect B3, a method includes the method of any B aspect, further including: delivering cardiac therapy to or sensing electrical activity of the left atrium (LA); and delivering cardiac therapy to or sensing electrical activity of the left ventricle (LV).

In illustrative aspect B4, a method includes the method of any B aspect, further including advancing the distal portion of the first implantable lead to the location to seal the puncture through the vessel wall.

In illustrative aspect B5, a method includes the method of any B aspect, wherein the first electrode is implanted after the second electrode is implanted.

In illustrative aspect C1, a method includes implanting a first implantable lead to implant a first electrode of the first implantable lead at a first implantation site in the coronary sinus or a coronary vein of a patient's heart. The method also includes advancing a second implantable lead having a second electrode guided by a distal portion of the first implantable lead to a second implantation site distal to the first electrode in a left ventricular (LV) myocardium of the patient's heart. The method further includes implanting the second electrode at the second implantation site.

In illustrative aspect C2, a method includes the method of any C aspect, wherein the first electrode is implanted in the coronary sinus or a coronary vein of the patient's heart and the second electrode is implanted in an LV myocardial wall of the patient's heart distal to the first electrode.

In illustrative aspect C3, a method includes the method of any C aspect, further including: delivering cardiac therapy to or sensing electrical activity of the left atrium (LA); and delivering cardiac therapy to or sensing electrical activity of the left ventricle (LV).

In illustrative aspect D1, a system includes a first implantable lead having a distal portion and a first electrode coupled to the distal portion of the first implantable lead. The first electrode is configured to be implanted at a first implantation site in the coronary sinus or a coronary vein of a patient's heart. The system also includes a second implantable lead having a distal portion and a second electrode coupled to the distal portion of the second implantable lead. The second electrode is configured to be implanted at a second implantation site distal to the first electrode in the patient's heart. The distal portion of the second implantable lead is guided by the distal portion of the first implantable lead to the first implantation site.

In illustrative aspect D2, a system includes the system of any D aspect, wherein the first electrode is implantable to deliver cardiac therapy to or sense electrical activity of the left atrium (LA) and the second electrode is implantable to deliver cardiac therapy to or sense electrical activity of the left ventricle (LV).

In illustrative aspect D3, a system includes the system of any D aspect, wherein both the first electrode and the second electrode are configured to provide pacing pulses.

In illustrative aspect D4, a system includes the system of aspect D1 or D2, wherein the first electrode is configured to only sense electrical activity and the second electrode is configured to provide pacing pulses.

In illustrative aspect D5, a system includes the system of any D aspect, wherein the second implantable lead includes a plurality of second electrodes configured to deliver cardiac therapy to or sense electrical activity of the left ventricle (LV).

In illustrative aspect D6, a system includes the system of any D aspect, wherein after the first or second electrode is implanted, the other of the first or second electrode is translatable relative to the implanted first or second electrode.

In illustrative aspect D7, a system includes the system of any D aspect, wherein the first or second electrode is configured to deliver electrical pulses to test the first or second implantation site.

In illustrative aspect D8, a system includes the system of any D aspect, wherein the second implantable lead is freely rotatable relative to the first implantable lead to facilitate implantation.

In illustrative aspect D9, a system includes the system of any D aspect, wherein the first electrode is implantable before or after the second electrode is implanted.

In illustrative aspect D10, a system includes the system of any D aspect, wherein the second implantable lead is at least partially receivable into a lumen of the first implantable lead while advancing the second implantable lead to the second implantation site.

In illustrative aspect D11, a system includes the system of any D aspect, wherein a sealing element is coupled between the first or second implantable lead to seal a distal end of the first implantable lead from fluid ingress.

In illustrative aspect D12, a system includes the system of any D aspect, wherein the first implantable lead includes a first fixation element formed integrally or separately from the first electrode.

In illustrative aspect D13, a system includes the system of any D aspect, wherein the second implantable lead includes a second fixation element formed integrally or separately from the second electrode.

In illustrative aspect D14, a system includes the system of any D aspect, wherein the first implantation site is in the great cardiac vein of the patient's heart.

In illustrative aspect D15, a system includes the system of any D aspect, wherein the second implantation site is in a coronary vein of the patient's heart.

In illustrative aspect D16, a system includes the system of any aspect D1-D14, wherein the second implantation site is in the left ventricular (LV) myocardium of the patient's heart.

In illustrative aspect D17, a system includes the system of any aspect D1-D14, wherein the second implantation site is in the myocardium of the patient's heart outside of the coronary sinus and the coronary veins.

In illustrative aspect D18, a system includes the system of aspect D16 or D17, further including a puncture element configured to puncture through a vessel wall at a location along the coronary sinus or the coronary vein into the pericardial cavity of the patient's heart.

In illustrative aspect D19, a system includes the system of aspect D18, wherein the puncture element is a microneedle configured to create a micropuncture in the vessel wall.

In illustrative aspect D20, a system includes the system of aspect D18 or D19, wherein the puncture element extends distally from a guidewire receivable within a lumen of the first implantable lead.

In illustrative aspect D21, a system includes the system of any aspect D18-D20, wherein a structure of the first implantable lead is configured to seal the puncture when the first electrode is implanted.

In illustrative aspect D22, a system includes the system of any D aspect, wherein the first electrode is proximal to a distal end of the first implantable lead.

Thus, various aspects of the LEAD-IN-LEAD SYSTEMS AND METHODS FOR CARDIAC THERAPY are disclosed. Although reference is made herein to the accompanying set of drawings that form part of this disclosure, one of at least ordinary skill in the art will appreciate that various adaptations and modifications of the aspects or features described herein are within, or do not depart from, the scope of this disclosure. For example, aspects of the disclosure described herein may be combined in a variety of ways with each other. Therefore, it is to be understood that, within the scope of the appended claims, the claimed invention may be practiced other than as explicitly described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device. In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer). Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements. All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out functionality.

The term “configured to” refers to an element with suitable structure to carry out a particular function. A suitable structure may be selected by a person having the benefit of this disclosure and at least ordinary skill in the art. As used herein, the term “configured to” may be used interchangeably with the terms “adapted to” or “structured to” unless the content of this disclosure clearly dictates otherwise.

Terms related to orientation, such as “proximal,” “distal,” “above,” or “below,” are used to describe relative positions of components and are not meant to limit the orientation of the embodiments contemplated.

Reference to “one embodiment,” “an embodiment,” “certain embodiments,” or “some embodiments,” etc., means that a particular feature, configuration, composition, or characteristic described in connection with the LEAD-IN-LEAD is included in at least one aspect of the disclosure. Thus, the appearances of such phrases in various places throughout are not necessarily referring to the same lead-in-lead of the disclosure. Furthermore, the particular features, configurations, compositions, or characteristics may be combined in any suitable manner in one or more lead-in-lead.

As used in this specification and the appended claims, the singular forms “a,” “an,” and “the” encompass embodiments having plural referents, unless the content clearly dictates otherwise. As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

1. A method comprising:
implanting a first implantable lead to implant a first electrode of the first implantable lead at a first implantation site in the coronary sinus or a coronary vein of a patient's heart;
advancing a second implantable lead comprising a second electrode guided by a distal portion of the first implantable lead to a second implantation site distal to the first electrode in a coronary vein of the patient's heart; and
implanting the second electrode at the second implantation site.

2. The method of claim 1, wherein the first electrode is implanted in the coronary sinus or a coronary vein of the patient's heart and the second electrode is implanted in a coronary vein of the patient's heart distal to the first electrode.

3. The method of claim 1 or 2, further comprising:
delivering cardiac therapy to or sensing electrical activity of the left atrium (LA); and
delivering cardiac therapy to or sensing electrical activity of the left ventricle (LV).

4. A method comprising:
advancing a second implantable lead comprising a second electrode guided by a distal portion of a first implantable lead comprising a first electrode to a location in the coronary sinus or a coronary vein of a patient's heart;
implanting the first electrode at a first implantation site in the coronary sinus or a coronary vein of the patient's heart;
puncturing through a vessel wall at the location in the coronary sinus or the coronary vein into the pericardial cavity of the patient's heart;
advancing the second implantable lead through the puncture in the vessel wall into the pericardial cavity; and
implanting the second electrode into the myocardium of the patient's heart from the pericardial cavity at a second implantation site distal to the first implantation site.

5. The method of claim 4, wherein the first electrode is implanted in the coronary sinus or a coronary vein of the patient's heart and the second electrode is implanted into the myocardium of the patient's heart outside of the coronary sinus or the coronary veins distal to the first electrode.

6. The method of claim 4 or 5, further comprising:
delivering cardiac therapy to or sensing electrical activity of the left atrium (LA); and
delivering cardiac therapy to or sensing electrical activity of the left ventricle (LV).

7. The method of claim 4, further comprising advancing the distal portion of the first implantable lead to the location to seal the puncture through the vessel wall.

8. The method of claim 4, wherein the first electrode is implanted after the second electrode is implanted.

9. A method comprising:
implanting a first implantable lead to implant a first electrode of the first implantable lead at a first implantation site in the coronary sinus or a coronary vein of a patient's heart;
advancing a second implantable lead comprising a second electrode guided by a distal portion of the first implantable lead to a second implantation site distal to the first electrode in a left ventricular (LV) myocardium of the patient's heart; and
implanting the second electrode at the second implantation site.

10. The method of claim 9, wherein the first electrode is implanted in the coronary sinus or a coronary vein of the patient's heart and the second electrode is implanted in an LV myocardial wall of the patient's heart distal to the first electrode.

11. The method of claim 9, further comprising:
delivering cardiac therapy to or sensing electrical activity of the left atrium (LA); and
delivering cardiac therapy to or sensing electrical activity of the left ventricle (LV).

12. A system comprising:
a first implantable lead comprising a distal portion and a first electrode coupled to the distal portion of the first implantable lead, wherein the first electrode is configured to be implanted at a first implantation site in the coronary sinus or a coronary vein of a patient's heart; and
a second implantable lead comprising a distal portion and a second electrode coupled to the distal portion of the second implantable lead, wherein the second electrode is configured to be implanted at a second implantation site distal to the first electrode in the patient's heart, wherein the distal portion of the second implantable lead is guided by the distal portion of the first implantable lead to the first implantation site.

13. The system according to claim 12, wherein the first electrode is implantable to deliver cardiac therapy to or sense electrical activity of the left atrium (LA) and the second electrode is implantable to deliver cardiac therapy to or sense electrical activity of the left ventricle (LV).

14. The system according to claim 12, wherein both the first electrode and the second electrode are configured to provide pacing pulses.

15. The system according to claim 12, wherein the first electrode is configured to only sense electrical activity and the second electrode is configured to provide pacing pulses.

16. The system according to claim 12, wherein the second implantable lead comprises a plurality of second electrodes configured to deliver cardiac therapy to or sense electrical activity of the left ventricle (LV).

17. The system according to claim 12, wherein after the first or second electrode is implanted, the other of the first or second electrode is translatable relative to the implanted first or second electrode.

18. The system according to claim 12, wherein the first or second electrode is configured to deliver electrical pulses to test the first or second implantation site.

19. The system according to claim 12, wherein the second implantable lead is freely rotatable relative to the first implantable lead to facilitate implantation.

20. The system according to claim 12, wherein the first electrode is implantable before or after the second electrode is implanted.

21. The system according to claim 12, wherein the second implantable lead is at least partially receivable into a lumen of the first implantable lead while advancing the second implantable lead to the second implantation site.

22. The system according to claim 12, wherein a sealing element is coupled between the first or second implantable lead to seal a distal end of the first implantable lead from fluid ingress.

23. The system according to claim 12, wherein the first implantable lead comprises a first fixation element formed integrally or separately from the first electrode.

24. The system according to claim 12, wherein the second implantable lead comprises a second fixation element formed integrally or separately from the second electrode.

25. The system according to claim 12, wherein the first implantation site is in the great cardiac vein of the patient's heart.

26. The system according to claim 12, wherein the second implantation site is in a coronary vein of the patient's heart.

27. The system according to claim 12, wherein the second implantation site is in the left ventricular (LV) myocardium of the patient's heart.

28. The system according to claim 12, wherein the second implantation site is in the myocardium of the patient's heart outside of the coronary sinus and the coronary veins.

29. The system according to claim 27 or 28, further comprising a puncture element configured to puncture through a vessel wall at a location along the coronary sinus or the coronary vein into the pericardial cavity of the patient's heart.

30. The system according to claim 29, wherein the puncture element is a microneedle configured to create a micropuncture in the vessel wall.

31. The system according to claim 29, wherein the puncture element extends distally from a guidewire receivable within a lumen of the first implantable lead.

32. The system according to claim 29, wherein a structure of the first implantable lead is configured to seal the puncture when the first electrode is implanted.

33. The system according to claim 12, wherein the first electrode is proximal to a distal end of the first implantable lead.
